(19) United States
(12) Patent Application Publication
(10) Pub. No.: US 2010/0264327 A1
(43) Pub. Date: Oct. 21, 2010

(54) ARRANGEMENT FOR EXPANDING THE PARTICLE ENERGY DISTRIBUTION OF A PARTICLE BEAM

(76) Inventors: Marc-Oliver Bönig, Nurnberg (DE); Gerd Hein, Herzogenaurach (DE)

Correspondence Address: BRINKS HOFER GILSON & LIONE, P.O. BOX 10395, CHICAGO, IL, 60610, US

(21) Appl. No.: 12/755,742

(22) Filed: Apr. 7, 2010

(30) Foreign Application Priority Data
Apr. 15, 2009 (DE) 10 2009 017 440.0

(51) Int. Cl. G21K 1/00; A61N 5/10

(52) U.S. Cl. 250/396 R

(57) ABSTRACT

An arrangement for expanding the particle energy distribution of a particle beam is provided. The arrangement includes at least two ripple filters, which are arranged in series such that one ripple filter is behind another ripple filter in a radiation direction.

This application claims the benefit of DE 10 2009 017 440.0 filed Apr. 15, 2009, which is hereby incorporated by reference.

### BACKGROUND

The present embodiments relate to an arrangement for expanding the particle energy distribution of a particle beam.

In a particle therapy treatment (e.g., of cancers), a particle beam including, for example, protons or heavy ions (e.g., carbon ions) is generated in a suitable accelerator. The particle beam is generated in the accelerator and guided into a treatment room via an exit window. In one embodiment, the particle beam can be directed into different treatment rooms in alternation by the accelerator. In the treatment room, a patient who is to receive the therapy is positioned (e.g., on a patient examination table) and where appropriate, is immobilized.

A target region in the body of the patient (e.g., tissue such as a tumor) is irradiated layer by layer. Depending on the energy of the particle beam, the particle beam penetrates the tissue to different depths, with the result that the tissue can be subdivided into slice-shaped sections or layers of equal penetration depth. The focused particle beam is moved across the individual layers of the target region in a process referred to as “beam scanning,” such that a plurality of points within an individual layer which lie, for example, on a raster grid, are irradiated. Provided the radiation intensity or the energies are correctly chosen, regions with complex shapes can also be irradiated with precision. The arrangement of the layers and points that are to be irradiated is chosen such that the planned dose distribution can be achieved.

The energy that the particles possess is achieved using the accelerator, the particle beam being virtually monoenergetic. In this case, the monoenergetic particles deposit energy within a very small localized region along the radiation direction, inside what is termed the “Bragg peak.”

In particle therapy, the aim is to achieve a spatially homogeneous dose distribution, which requires a spatial expansion of the energy distribution of the particle beam. In this case, a Gaussian dose depth profile is desirable instead of the Bragg peak along the beam axis. A passive filter element (e.g., a ripple filter) is introduced into the beam path between the exit window and the patient. With the aid of the ripple filter, the desired expansion is achieved. In order to minimize side-effects, water-like materials are used as the material for the passive filter element. Plexiglas panels with grooves having a particular geometry that are introduced using a milling process may be used. A ripple filter such as this is described, for example, in the article titled “Design and construction of a ripple filter for a smoothed depth dose distribution in conformal particle therapy” by U. Weber and G. Kraft, Phys. Med. Biol. 44 (1999), 2765-2775.

Different geometries or milling contours of the grooves are used for different beam expansions. Given that the precision in the groove geometry is in the micrometer range and the filter size may be 20×20 cm, there are technical difficulties to be overcome in terms of precision and reproducibility in the production of the grooves. This applies most of all to filters for higher desired beam expansions, since the demands on the geometry of the grooves (e.g., the ratio of groove height to groove width) become more and more extreme. During the milling process of the relatively soft Plexiglas, undesirable interactions take place between the milling tool and the material, such as compressions and displacement. Consequently, the desired precision and reproducibility can be attained only with difficulty. Ripple filters for a beam expansion of 2 mm, 3 mm and 4 mm (hereinafter referred to as 2 mm, 3 mm and 4 mm ripple filters) are produced using milling. Instead of the Bragg peak, a Gaussian profile of the particle beam with a full width at half maximum of correspondingly 2 mm, 3 mm or 4 mm is established after the filter. While the 2 mm ripple filter can be manufactured with the desired precision and reproducibility, the 3 mm ripple filter and the 4 mm ripple filter are much more difficult to manufacture.

The problem of the high-precision manufacture of the ripple filters has been solved in the prior art through optimization of milling cutters and milling techniques, this being associated with high manufacturing costs for the milling cutter.

### SUMMARY AND DESCRIPTION

The present embodiments may obviate one or more of the drawbacks or limitations in the related art. For example, in one embodiment, a particle beam may be expanded cost-effectively and with a high degree of precision.

In one embodiment, an arrangement for expanding the particle energy distribution of a particle beam includes at least first and second ripple filters, which are arranged in series such that the first ripple filter is behind the second ripple filter in a radiation direction.

The present embodiments are based on the consideration that a filter having a desired high beam expansion that is technically difficult to manufacture may be realized using two or more easy-to-manufacture filters of low beam expansion. In this case, the at least first and second ripple filters are arranged in series, the first ripple filter behind the second ripple filter in the radiation direction, in order to modify the particle energy distribution in stages.

Filters having a higher beam expansion (e.g. 4 mm or 5 mm) may be implemented using the plural arrangement of ripple filters (e.g., 2 mm and 3 mm ripple filters). With such an arrangement, the greater quantity of material that is guided into the particle beam path leads to an increased energy loss. However, the increased energy loss is constant and may be corrected by performing the particle therapy with a higher beam energy.

The first ripple filter has a first beam expansion property such that the particle beam passing through the first ripple filter is expanded by a first amount. The second ripple filter has a second beam expansion property such that the particle beam passing through the second ripple filter is expanded by a second amount. The first beam expansion property and the second beam expansion property may be the same or may be different. The first ripple filter and the second ripple filter may be arranged in series such that a combined beam expansion property of the first and second ripple filters is higher than the first beam expansion property or the second beam expansion property. However, the first ripple filter and the second ripple filter may be arranged in series such that the combined beam expansion property is different from the sum of the first beam expansion property and the second beam expansion property.

In one embodiment of a series connection of two filters that are aligned equally, grooves of the two filters are aligned with one another. In one embodiment, a simplification of the arrangement of the individual ripple filters is provided in that the ripple filters are rotated relative to each other. As a result of the rotating of the ripple filters relative to each other a plurality of filters may be combined and positioned one behind the other.

In one embodiment, two ripple filters, which are rotated 90° relative to each other, are provided. Manufacturing tolerances of the individual filters are statistically equalized since the groove structures of the two ripple filters are orthogonally superimposed.

In one embodiment, the ripple filters used are configured for a beam expansion of 2 mm, 3 mm, or 2 mm and 3 mm. Ripple filters of this type may be manufactured with high precision and reproducibility using conventional production methods, so a greater expansion of the particle beam is achieved at low cost and with little overhead by the connection of the ripple filters in series.

In one embodiment, the two ripple filters used are a filter for a beam expansion of 2 mm and, connected downstream of the 2 mm filter, a filter for a beam expansion of 3 mm. As a result of this arrangement, which is particularly simple to construct and reproduce, a beam expansion of 4 mm is achieved.

In one embodiment, the two ripple filters are arranged one behind the other at a maximum spacing of about 1 m apart. The ripple filter for the beam expansion of 3 mm is positioned at a maximum of about 1 m behind the ripple filter for the beam expansion of 2 mm. In order to reduce the amount of space for the arrangement, the distance between the two filters may be minimized. For example, the two filters may be adhered, one on top of the other.

In one embodiment, the at least two ripple filters are plates having a periodic structure including fine grooves. Because ripple filters for a beam expansion of 2 mm or 3 mm are used in one embodiment described above, the machining of the plates in order to produce the fine grooves does not constitute a great overhead. The desired shape of the grooves may be produced with a mechanical precision of approximately 5 μm to 10 μm.

In one embodiment, the at least two ripple filters are made of polymethylmethacrylate (PMMA).

In one embodiment, a particle therapy system having an arrangement in accordance with one of the embodiments described above is provided.

In one embodiment, a method for expanding the particle energy distribution of a particle beam, where at least two ripple filters are connected in series, one ripple filter behind another ripple filter in the radiation direction, is provided. The advantages and preferred embodiments presented in relation to the arrangement are to be applied analogously to the method.

### BRIEF DESCRIPTION OF THE DRAWINGS

An exemplary embodiment of the invention is explained below with reference to a drawing, in which:

FIG. 1 shows a particle therapy system,

FIG. 2 shows the structure of one embodiment of a ripple filter,

FIG. 3 schematically shows one embodiment of an arrangement of two ripple filters in a radiation direction, and

FIG. 4 shows a plot of the results of a measurement using the arrangement according to FIG. 3.

### DETAILED DESCRIPTION OF THE DRAWINGS

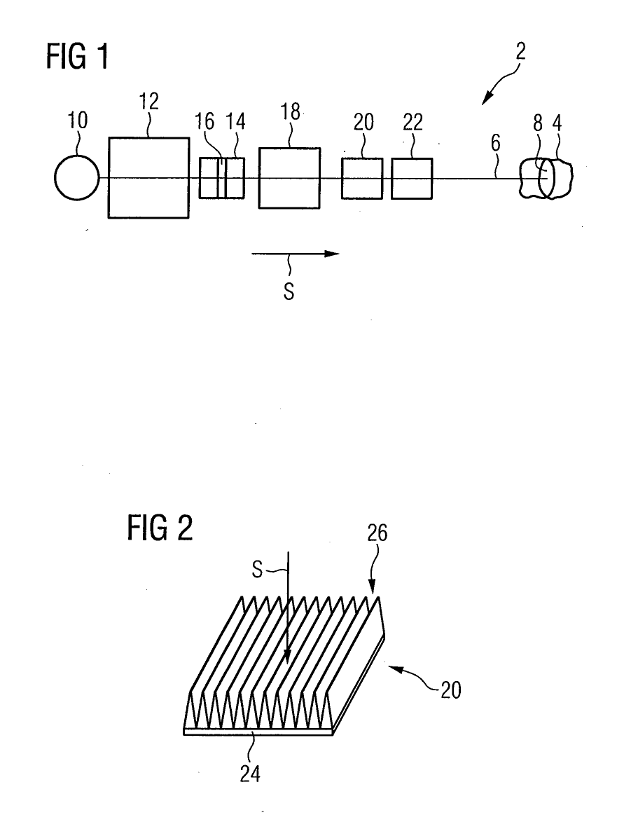

FIG. 1 shows a particle therapy system 2. The particle therapy system 2 is used to irradiate a tumor tissue 4 in stages with the aid of a particle beam 6, a disk-shaped section 8 (i.e., a layer 8) of the tumor 4 being treated at each stage. The particle beam 6 is generated in an accelerator 10, which is controlled by a control unit 12. The accelerator 10 delivers the particles at an energy that is required for the layer 8, which is currently to be irradiated. The energy may lie in the two- to three-digit MeV range (e.g., in the range of 100 MeV). The control unit 12 includes a raster-scan device (not shown here), which deflects the particle beam 6 both in the horizontal direction and in the vertical direction in order to scan the tumor tissue within the layer 8. For this purpose, the raster-scan device includes two pairs of magnets, for example.

During the irradiation of the tumor 4 of a patient (not shown), the particle beam 6 is set to a high energy level by way of the accelerator 10 so that the particle beam 6 reaches the peripheral region of the tumor 4 (e.g., shown on the right in FIG. 1). In this process, a plurality of scanning points of the reached layer 8 of the tumor 4 are selectively irradiated.

The particle beam 6 also runs through an energy modulator 14 arranged in the beam path. The energy modulator 14 includes an absorber element 16, which absorbs part of the energy of the particle beam 6 when the particle beam 6 passes through the material of the absorber element 16 and consequently, limits the range of the particles. The particle beam 6 also passes through a monitoring system 18, which may be, for example, a particle counter. The particle dose deposited in the region of the tumor 4 is dependent on the number of particles present in the particle beam 6. During the irradiation process, the number of particles acting on the tumor 4 is determined using the monitoring system 18. When the desired number of particles is reached in a scanning point, a signal is output to the control unit 12, which controls the raster-scan device for the purpose of aligning the particle beam 6 onto a next scanning point.

The particle therapy system 2 also includes a device for modifying the spatial energy distribution of the particle beam 6 further along the beam. In one embodiment, the device for modifying the spatial energy distribution of the particle beam 6 may include a ripple filter 20 for expanding the particle beam 6 in a radiation direction S and a focusing device 22 for expanding the particle beam 6 radially relative to the radiation direction S.

FIG. 2 schematically shows one embodiment of the structure of a ripple filter 20 for a particle energy expansion of 2 mm. The ripple filter 20 may be, for example, manufactured from a thin Plexiglas plate 24 (PMMA), which is approximately 200×200×2 mm in size. The ripple filter 20 shown in FIG. 1 has a periodic structure formed from a plurality of fine and precisely milled grooves 26 having a precise cross-sectional geometry.

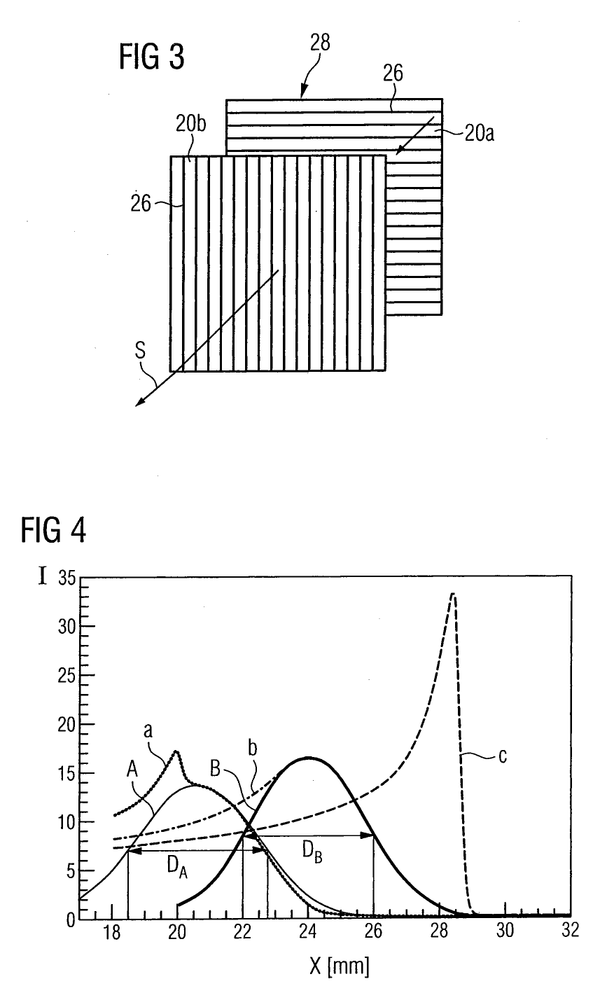

In order to achieve an expansion of the particle beam 6 of 4 mm, for example, an arrangement 28 of two ripple filters 20a, 20b, positioned such that a first ripple filter 20a is behind a second ripple filter 20b, is introduced in the radiation direction S, as shown in FIG. 3. In one embodiment, the first ripple filter 20a in the radiation direction S is a filter for expanding the particle beam 6 by 2 mm, and the second ripple filter 20b is connected downstream of the first ripple filter 20a expands the particle beam 6 by 3 mm. The two ripple filters 20a, 20b are positioned approximately 1 m apart from each other. The two ripple filters 20a, 20b are also rotated through 90° relative to each other, such that the plurality of grooves 26 of the two ripple filters 20a, 20b run orthogonally to each other.

FIG. 4 shows a plot of the results of a measurement of the expansion of the energy distribution of the particle beam 6 using the arrangement 28 shown in FIG. 3. In FIG. 4, a relative dose I is plotted against a penetration depth X of the particle beam 6 in millimeters. The measurement using the arrangement 28 is indicated by the curve “b,” which has a Gaussian profile. The measurement using the two series-connected ripple filters 20a, 20b was performed at a beam energy of 108.53 MeV. This increased beam energy was chosen in order to compensate for the additional energy loss due to the second ripple filter 20b.

FIG. 4 also shows a plot of the results of a measurement performed at a beam energy of 88.83 MeV using a 4 mm ripple filter for purposes of comparison. This curve is identified by “a” in FIG. 4.

In addition, FIG. 4 shows a plot of the results of a measurement without the two ripple filters 20a, 20b as a reference measurement. The reference measurement shows the undisturbed Bragg peak, which is identified by “c”.

A Gaussian function, represented by the curves A and B, is adapted in each case to the two curves a and b, respectively, of the measurements taken using the 4 mm ripple filter and using the combination of the 2 mm ripple filter with the 3 mm ripple filter.

The broadening of the Bragg peak when using the two ripple filters of 2 mm and 3 mm rotated through 90° results in an expansion which essentially is comparable in size to that of a single 4 mm ripple filter. This is recognizable with reference to the full widths at half maximum (FWHM) of the curves A and B: the full width at half maximum D_{A} of the curve A when using the 4 mm filter approximately corresponds to the full width at half maximum D_{B} of the curve B when using the combination of the 2 mm ripple filter with the 3 mm ripple filter and within the scope of the evaluation precision, lies at approximately 4 mm. With the curve “b,” no erroneous overshoot, as in the case of the curve “a,” can be observed. This shows experimentally that the series connection is more tolerant with regard to manufacturing defects.

Because the two ripple filters are rotated through 90°, the manufacturing defects of the two filters cancel one another out. Consequently, the manufacturing tolerances for the individual filter elements may be greater. Since a greater total thickness is present due to the use of the two ripple filters, the additional thickness was compensated for by a higher beam energy.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. An arrangement for expanding a particle energy distribution of a particle beam, the arrangement comprising at least first and second ripple filters, which are arranged in series such that the first ripple filter is behind the second ripple filter in a radiation direction.

2. The arrangement as claimed in claim 1, wherein the first and second ripple filters are rotated relative to one another.

3. The arrangement as claimed in claim 2, wherein the first and second ripple filters are rotated through 90° relative to one other.

4. The arrangement as claimed in claim 1, wherein the first and second ripple filters are configured to expand the particle energy distribution of the particle beam 2 mm, 3 mm, or 2 mm and 3 mm.

5. The arrangement as claimed in claim 3, wherein the first ripple filter expands the particle energy distribution of the particle beam 2 mm, and the second ripple filter is connected downstream of the first ripple filter and expands the particle energy distribution of the particle beam 3 mm.

6. The arrangement as claimed in claim 2, wherein the first and second ripple filters are arranged such that the first ripple filter is behind the second ripple filter at a maximum spacing of about 1 m apart.

7. The arrangement as claimed in claim 1, wherein the first and second ripple filters are plates having a periodic structure consisting of fine grooves.

8. The arrangement as claimed in claim 1, wherein the first and second ripple filters are made of polymethylmethacrylate.

9. A particle therapy system comprising:
an arrangement for expanding a particle energy distribution of a particle beam, the arrangement comprising two ripple filters, which are arranged in series such that one ripple filter is behind another ripple filter in a radiation direction.

10. A method for expanding the particle energy distribution of a particle beam, wherein two ripple filters are connected in series such that a first ripple filter is behind a second ripple filter in a radiation direction.

11. The arrangement as claimed in claim 2, wherein the first and second ripple filters are configured to expand the particle energy distribution of the particle beam 2 mm, 3 mm, or 2 mm and 3 mm.

12. The arrangement as claimed in claim 4, wherein the first and second ripple filters are arranged such that the first ripple filter is behind the second ripple filter at a maximum spacing of about 1 m apart.

13. The arrangement as claimed in claim 5, wherein the first and second ripple filters are arranged such that the first ripple filter is behind the second ripple filter at a maximum spacing of about 1 m apart.

14. The arrangement as claimed in claim 2, wherein the first and second ripple filters are plates having a periodic structure consisting of fine grooves.

15. The arrangement as claimed in claim 3, wherein the first and second ripple filters are plates having a periodic structure consisting of fine grooves.

16. The arrangement as claimed in claim 4, wherein the first and second ripple filters are plates having a periodic structure consisting of fine grooves.

17. The arrangement as claimed in claim 6, wherein the first and second ripple filters are plates having a periodic structure consisting of fine grooves.

18. The arrangement as claimed in claim 2, wherein the first and second ripple filters are made of polymethylmethacrylate.

19. The particle therapy system as claimed in claim 9, wherein the two ripple filters are rotated relative to one another.

20. The particle therapy system as claimed in claim 9, wherein the two ripple filters are plates having a periodic structure consisting of fine grooves.
